(19) United States
(12) Patent Application Publication
(10) Pub. No.: US 2003/0194383 A1
(43) Pub. Date: Oct. 16, 2003

(54) Teeth whitening system

(75) Inventors: James Louis Gentile, Orange, CT (US); Christine Watson Ryles, Milford, CT (US); David Robert Williams, Monroe, CT (US); Alexander George Ziemkiewicz, Shelton, CT (US)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 10/273,281

(22) Filed: Oct. 17, 2002

Related U.S. Application Data
(60) Provisional application No. 60/372,647, filed on Apr. 15, 2002.

(51) Int. Cl. A61K 7/20; A61C 5/00

(52) U.S. Cl. 424/53; 433/217.1

(57) ABSTRACT

A kit and method for whitening teeth is provided which utilizes a U-shaped dental tray fittable over a person's teeth and a bleaching composition formed from a combination of first and second compositions which prior to use are stored separately from one another. The first composition includes a peroxide and the second composition includes an alkaline material. At least one of the first and second compositions includes an anionic surfactant; the latter permits the bleaching composition to better penetrate all surfaces surrounding the teeth.

### BACKGROUND OF THE INVENTION

1. Field of the Invention

The invention concerns a kit and method for whitening teeth through application of an implement delivering a bleaching composition.

2. The Related Art

Sparkling white teeth enhance anyone's smile. Perhaps for this reason people have been drawn to systems promising whiter teeth. Dentists have taken a lead in meeting this consumer demand. Veneers often in the form of caps have been placed over teeth or these have been subjected to chemical bleaching. Placement of veneers or caps is quite expensive. In-office chemical bleaching procedures often involve carefully placing a hydrogen peroxide solution (typically 30% active) on the teeth, protecting the sensitive soft tissues with rubber dam, and applying heat or light to the solution. Treatments typically last almost an hour, and multiple appointments are necessary to achieve any significant change. Cost for these treatments is quite significant.

The need for a dental professional results from failure by traditional toothpastes to achieve sufficiently consumer perceivable levels of whitening. One of the better toothpaste formulations is described in U.S. Pat. No. 5,902,568 (Ryles et al.). Therein a method for whitening teeth is described utilizing an oral composition with first and second bicarbonate paste and peroxide get streams stored separate from one another prior to time of use. The oral composition has a pH from 9.0 to 12. A dual chambered dispensing pump delivers each of the streams for mixing at point of use. Application is through brushing with a toothbrush.

The demand for more effective yet convenient in-home use has spawned a variety of bleaching kits. Generally, these utilize a peroxy formulated liquid or gel placed within a reservoir of a dental appliance and held against the teeth for a period of at least fifteen minutes.

Illustrative of this art is U.S. Pat. No. 5,076,791 (Madray, Jr.) disclosing a pre-formed stock tray of a thermoplastic material with a low-heat melt-index and a peroxide gel. The tray is custom fitted at home by the wearer, instead of having to go to a dentist. It is placed in boiling water for a few seconds to soften, snuggly pressed over the teeth to form an impression, and then removed as a molded tray. An oxidizing gel is placed inside the molded tray, which in turn is again placed over the teeth. Treatment is done several times throughout the day, and continued for 2-3 weeks.

U.S. Pat. No. 5,165,424 (Silverman) reports a similar procedure utilizing 10% carbamide peroxide as the active bleaching ingredient. U.S. Pat. No. 5,770,182 (Fischer) describes an improved result through use of carbamide peroxide in a high viscosity matrix formulation that provides sustained release of the active. A high concentration of carboxypolymethylene is utilized to impart the very high viscosity.

Two compartment separate peroxide and alkaline material streams have been utilized with mechanical implements in the bleaching of teeth. For instance, U.S. Pat. No. 5,928,628 (Pellico) employs a gel of hydrogen peroxide structured by a polyoxyethylene polyoxypropylene block copolymer known as Poloxamer® 407 formulated as an acidic composition. An alkaline gel is formed with a similar structurant but without peroxide. Contact of the two separated compositions at the point of use generates active bleaching on the teeth.

U.S. Pat. No. 4,983,380 (Yarbrough) discloses a method by which a mucoprotectant gelatin forms a protective coating on the mucous membranes of the mouth while hydrogen peroxide bleaching occurs against the teeth. The mucoprotectant shields the gums from damage. Sodium stearate is employed as the mucoprotectant. When contacted with the peroxide, sodium stearate breaks down releasing stearic acid, the latter acting as a protective coating against the gums.

A problem of the known art is that the whitening effect can often not be achieved along the margins of the teeth. These areas are very difficult to reach by the gels and pastes which have characterized the art.

Accordingly, it is an advantage of the present invention to provide a method and kit whereby even the margins between teeth can be treated in a convenient manner.

### SUMMARY OF THE INVENTION

A kit is provided for whitening teeth, the kit including:

(i) a U-shaped dental tray fittable over a person's teeth;

(ii) a bleaching composition formed from combining a first and second composition, the compositions being separately stored and including:

(a) a first composition including from about 0.1 to about 30% of a peroxide in a cosmetically acceptable carrier;

(b) a second composition including from about 0.1 to about 30% of an alkaline material in a cosmetically acceptable carrier, wherein at least one of the first and second compositions comprises an anionic surfactant;

(iii) written instructions for placing the bleaching composition within the U-shaped tray and applying the tray over the person's teeth for a length of time sufficient to whiten teeth.

Furthermore, a method is provided for whitening teeth which includes:

(a) providing a U-shaped dental tray fittable over a person's teeth;

(b) placing a bleaching composition within the U-shaped tray, the bleaching composition formed from combination of a first and second composition, the first and second compositions being separately stored and including:

(a) a first composition including from about 0.1 to about 30% of a peroxide in a cosmetically acceptable carrier;

(b) a second composition including from about 0.1 to about 30% of an alkaline material in a cosmetically acceptable carrier, wherein at least one of the first and second compositions includes an anionic surfactant;

(c) applying the bleaching composition filled U-shaped tray over the person's teeth for a length of time sufficient to whiten the teeth.

### BRIEF DESCRIPTION OF THE DRAWING

Further features and advantages of the present invention will become more readily apparent from consideration of the following drawing in which:

FIG. 1 is a top plan perspective view of a dental tray according to the present invention; and

FIG. 2 is a bottom plan view of the tray shown in FIG. 1.

### DETAILED DESCRIPTION OF THE INVENTION

Now there has been found an improved system for whitening teeth. The system employs a U-shaped dental tray fittable over a person's teeth by which a special bleaching composition is suspended in contact with those teeth. This composition delivers peroxide through a less viscous and more penetrating formulation than previously known systems. The lower viscosity and foaming characteristics allow penetration of the composition even between teeth and contact with all crevices. The improved result is achieved by formulating an anionic surfactant within the bleaching composition. The resultant foamed bleaching composition can evenly flow around tooth surfaces yet does not readily drain away. Furthermore, the system demineralizes teeth less than others and may result in less tooth sensitivity.

According to the present invention, the bleaching composition is formed from a combination of two separately stored first and second compositions. The first is an acidic peroxide containing composition, preferably in gel form. The second composition is an alkaline one, preferably in paste form. These are each stored separately prior to use.

Packaging may include either two separate tubes, dual side-by-side separate chambered squeeze tubes, dual-compartmented dispensing pumps, squeezable tube with concentric inner tube, or single chambered tube and dispensers wherein each of the compositions lies adjacent one another. Delivery may also be through a squeezable plastic tube containing two discrete compositions stored in separate areas one above the other in a manner similar to systems for striping toothpaste. Illustrative of this technology is U.S. Pat. No. 4,211,341 (Weyn) hereinafter incorporated by reference.

Separate storage of the two compositions of this invention may be accomplished through a dual compartment dispenser. U.S. Pat. No. 4,687,663 (Schaeffer) discloses a dual-compartment package respectively storing a peroxide gel and a bicarbonate paste. Pump packaging with multiple compartments is described in U.S. Pat. No. 5,038,963 (Pettengill et al.) and U.S. Pat. No. 5,020,694 (Pettengill), all of which are herewith incorporated by reference.

The dispenser may be a system in the form of two individual tubes quite separate from one another but packaged within a kit. Alternatively, the tubes may be in a side-by-side arrangement with a common exit nozzle as in U.S. Pat. No. 5,318,203 (Iaia et al.)

Delivery may also be from a single chambered tube except that each of the two compositions are semi solid strips positioned side-by-side touching but not mixing with one another. The relatively high viscosity of the products prevents any substantial transference of either pH change or components between the two strips. Illustrative of this technology is a U.S. product sold by Colgate® under the Baking Soda & Peroxide brand.

Still another method of delivery may be a single composition such as a paste or gel housing an alkaline environment. Peroxide such as calcium peroxide may be dispersed throughout the alkaline composition yet separated from contact by an encapsulating coating. Activation occurs within the tray in the mouth through the presence of water or saliva which penetrates the encapsulating coating releasing peroxide to interact with the alkaline environment.

The bleaching composition which is a combination of the first and second compositions admixed together advantageously exhibits a pH range from about 7 to about 11, preferably from 9 to 10.5, optimally from 9.2 to 10.0.

First compositions of the present invention will include a peroxide. The peroxide should be capable of generating hydrogen peroxide such as sodium perborate, sodium persilicate, sodium percarbonate, sodium perphosphate, calcium peroxide, sodium peroxide, magnesium persulphate, carbamide peroxide and aqueous hydrogen peroxide. Most preferred is an aqueous solution of hydrogen peroxide. Amounts of the peroxide may range from about 0.1 to about 30%, preferably from about 0.5 to about 10%, optimally from about 1 to about 5% by weight of the total oral composition.

The peroxide containing composition may either be a liquid, paste or gel, preferably the latter. When a gel, water will be present in amounts ranging from about 5 to about 70%, preferably from about 10 to about 55%, optimally between about 20 to about 40% by weight of the first composition.

A variety of other ingredients normally present in dentifrices can be selected for the first and second compositions of the present invention. When the peroxide composition is intended as a get, it is usual to employ with the water a thickening agent that is a cross-linked acrylic polymer. Alternatively and most preferably, gets can be structured with a polyoxyethylene-polyoxypropylene copolymer. Commercially, copolymers are available from the BASF Corporation under the trademark Pluracare® F88, F99, F108 and F127. Most preferred is Pluracare® F127 (more commonly described by its CTFA name, Poloxamer 407) which has a molecular weight ranging from 10,000 to 15,000, and containing 70% of the hydrophilic polyoxyethylene moiety. Amounts of the copolymer can range anywhere from 18-25% by weight, preferably between 19 and 24%, by weight of the peroxide containing stream.

Advantageously, glycerol may also be present in the peroxide composition in an amount from about 15 to about 60%, preferably in an amount greater than 30% but less than 50%, optimally between 35 to 45% by weight of the peroxide containing stream.

A low pH, preferably a pH no higher than 6, optimally less than 3.0, and generally from about 2.5 to 2.9 is maintained for peroxide gels. Acidification is accomplished through use of an inorganic or organic acid.

Acidity in the first composition may be achieved by formulating in addition to a peroxide such as hydrogen peroxide, inorganic acids which may include phosphoric, hydrochloric, nitric or boric acids, and organic C₂-C₂₀ carboxylic acids such as citric, malic, lactic, alginic, succinic, tartaric and ascorbic acids. Soluble salts may also be employed such as potassium bitartrate, sodium acid citrate, acid phosphate and pyrophosphate salts such as monosodium phosphate and disodium pyrophosphate.

The second composition of the invention will be an alkaline one, preferably an opaque paste. Typically the alkaline material may be selected from sodium bicarbonate, sodium carbonate, potassium bicarbonate, potassium carbonate, lithium carbonate, lithium bicarbonate, sodium hydroxide, triethanolamine, magnesium carbonate, potassium hydroxide and mixtures thereof. Most preferred is sodium carbonate.

The preferred embodiment utilizes a mixture of alkali metal (e.g. sodium or potassium) bicarbonate and carbonate. Most preferred are the sodium salts. The weight ratio of bicarbonate to carbonate may range from about 1:2 to about 1:100, preferably from about 1:4 to about 1:30, optimally from about 1:5 to about 1:10.

Normally, the alkaline material is included in the composition in an amount sufficient to provide a neutral or basic pH when the total bleaching composition is contacted with water. Thus, the pH of the second composition may range from about 8 to about 11, more preferably from about 9.5 to about 10.5, optimally from about 9.8 to about 10.2. Amounts of the alkaline material may range from about 0.1 to about 60%, preferably from about 0.5 to about 30%, more preferably from about 1 to about 20%, optimally from about 3 to about 15% by weight of the second composition.

Each of the first and second compositions will include a cosmetically acceptable carrier. The term “cosmetically acceptable carrier” will include such functional ingredients as water, humectants, abrasives, thickeners and combinations thereof. Total levels of these materials may range anywhere from about 1 to about 95%, preferably from about 20 to about 80%, optimally from about 30 to about 60% by weight of each composition.

Among the carriers, water when present may range in amount from about 1 to about 95%, preferably from about 15 to about 60%, optimally from about 30 to about 50% by weight of each of the compositions. Of course some formulations may be anhydrous.

Humectants are usually also present as one of the carriers. Illustrative of this category are sorbitol, maltitol, mannitol, glycerin, propylene glycol, polypropylene glycol, polyethyleneglycol, hydrogenated corn syrup, lactitol, xylitol and mixtures thereof. Amounts of the humectant may range from about 1 to about 60%, preferably from about 5 to about 50%, optimally from about 10 to about 40% by weight of each composition.

Abrasives are normally present in toothpastes and some gels. These may include sodium metaphosphate, dicalcium phosphate, calcium pyrophosphate, silica, alumina, chalk, insoluble bicarbonate salts, and mixtures thereof. Amounts of the abrasives may range from about 1 to about 80%, preferably from 5 to 50% by weight of each composition.

Thickeners are a further type of carrier which can be included in the compositions of this invention. Illustrative are hydroxy ethyl cellulose, methyl cellulose, ethyl cellulose, gum tragacanth, gum arabic, gum karaya, sodium alginate, carrageenan, guar, Irish moss, starch, modified starch, Carbomers (crosslinked acrylates) and mixtures thereof. Inorganic substances may also be suitable, especially silica aerogels and magnesium aluminum silicate (e.g. Veegum®). Amounts of the thickener may range from about 0.01 to about 30%, preferably from about 0.1 to about 20%, optimally from about 0.5 to about 15% by weight of a composition.

An important feature of the present invention is the presence of an anionic surfactant. Illustrative are sodium lauryl sulphate, sodium dodecylbenzene sulphonate, sodium lauryl sarcosinate, sodium or potassium tactylate, sodium cocoyl isethionate, sodium lauryl ether sulphate and combinations thereof. Most preferred is sodium lauryl sulphate and sodium lauryl ether sulphate.

Amounts of the anionic surfactant should be present at an effective level in the bleaching composition to reduce viscosity relative to a bleaching composition without the anionic surfactant. In particular, the anionic surfactant can be in either or both of the first and second compositions in an amount which delivers to the bleaching composition (mixture of first and second compositions) from about 0.01 to about 20%, preferably from about 0.1 to about 15%, more preferably from about 0.5 to about 10%, optimally from about 1 to about 5%, more optimally from greater than 1.5 to 3% by weight of the bleaching composition. Although usable, soaps such as alkali metal C₈-C₂₂ fatty acids, and particularly sodium stearate, may be excluded.

Flavors may also be present in the compositions. These flavors may be based on oils of spearmint and peppermint. Examples of other flavoring materials include menthol, clove, wintergreen, eucalyptus and aniseed. Flavors may range in concentration from about 0.01 to about 5% by weight of a composition.

Sweetening agents may also be included such as saccharin, sodium cyclamate, aspartame, ace-sulfame, xylitol and combinations thereof at levels from about 0.1 to about 10% by weight of a composition.

Gel compositions incorporating hydrogen peroxide may include a sequestering agent(s) such as a pyrophosphate or other phosphate for chelation of ferric/ferrous ion as well as other transition metal ions to enhance hydrogen peroxide stability. The sequestering agents may also be included within the paste compositions and are present in amounts from about 0.01 to about 20% by weight of a composition. Most preferred chelants are tetrasodium pyrophosphate, sodium tripolyphosphate and sodium hexametaphosphate. Other organic chelating agents such as sodium citrate and zinc citrate are also useful.

Other additives may also be incorporated such as preservatives, silicones, other synthetic or natural polymers such as Gantrez S97®, antitartar actives and antigingivitis actives. Among the antitartar agents are included zinc citrate, tetrasodium pyrophosphate, disodium pyrophosphate, dipotassium pyrophosphate, tetrapotassium pyrophosphate and mixtures thereof. Antigingivitis actives may include thymol, Triclosan, stannous gluconate and mixtures thereof. Amounts of each of the aforementioned ingredients will depend upon their function. Generally each of these substances wilt range in amounts from about 0.01 to about 20% by weight of a composition.

For anti-caries protection, a source of fluoride ion may be present in one or both the first and second compositions of the total bleaching composition. However, in whitening kits of this invention the fluoride is often best omitted. Fluoride sources include sodium fluoride, potassium fluoride, stannous fluoride, stannous monofluorophosphate and sodium monofluorophosphate. These sources when present should release anywhere from 25 to 3500 ppm of fluoride ion. Specific amounts of the anti-caries agent may range from 0.05 to 3% by weight, preferably from 0.2 to 0.6% by weight of the total bleaching composition.

Kits of the present invention besides including a U-shaped dental tray and the first and second compositions in a suitable dispensing package may also contain adjunct components. These may include a mouth rinse for preparing the enamel prior to application of the bleaching composition. A wooden or plastic paddle may be provided to assist in mixing of the bleaching composition and distributing same within the U-shaped tray. The mouth rinse can typically comprise water, ethyl alcohol, sorbitol, sodium lauryl sulphate, Polysorbate 20, flavor, sodium saccharin, sodium acetate, acetic acid and a colorant (e.g. FD&C Blue 1). An instruction guide may be provided with the kit with directions for utilizing the various components and may also include a shade guide to monitor the progress of changes in the tooth colors.

According to the system, a user may first be requested to rinse debris and plaque from teeth through a gargle with the mouth rinse. Thereafter, the first and second compositions are extruded from their separate chambers within a dual-chambered telescopic pump such as described in the Pettengill patents. The extruded first and second compositions are ribboned into a trough of a U-shaped dental tray. These compositions are intimately mixed with the aid of a paddle. During the mixing process, the viscosity of the bleaching composition decreases with foaming becoming prevalent. This composition is spread evenly within the trough. Thereafter the filled tray is applied to the teeth. Unlike some known kits which require the dental tray to be heated (e.g. in hot water) so that it can mold to the shape of a user's teeth, the present tray need not be so molded and may even preferably not be heated. By a loose fit of the tray to the teeth, there is opportunity for the reduced viscosity bleaching composition to penetrate all around the teeth including crevices and spaces between teeth. Application to the teeth is recommended for a period of from about 5 minutes to about 2 hours, preferably from about 10 to about 30 minutes, optimally about 15 minutes.

Bleaching compositions of the present invention may have a viscosity ranging from about 2,000 to about 100,000, preferably from about 4,000 to about 60,000, even more preferably from about 5,000 to about 10,000, optimally from about 6,000 to about 8,000 cps, measured on a Brookfield LV Viscometer employing a T-bar C at 50 rpm and at 20° C.

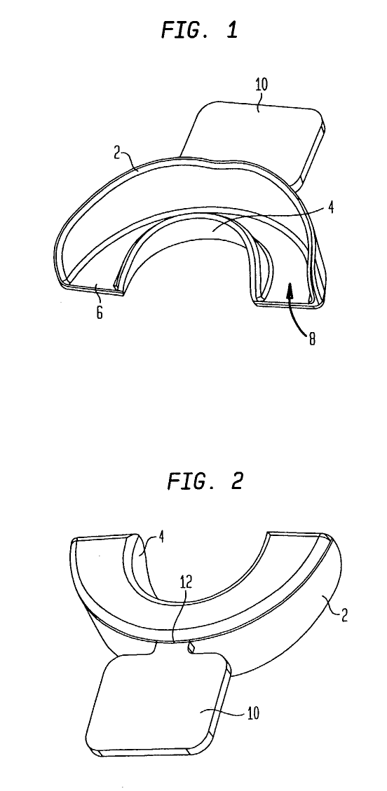

FIG. 1 illustrates a U-shaped dental tray according to the present invention. The tray includes an anterior curved wall 2 and a posterior curved walt 4 joined by a connector bridge 6. A U-shaped trough 8 is formed by the boundaries of the posterior curved wall, anterior curved wall and connector bridge.

FIG. 2 best illustrates a handle 10 joined to the anterior wall by a stem 12.

Advantageously the dental tray is manufactured as a unitary piece utilizing an elastomeric rubber material such as polyethylvinylacetate, low density polyethylene or high density polyethylene. Normally a kit will include two trays, one for the upper and another for the lower set of teeth.

Except in the operating and comparative examples, or where otherwise explicitly indicated, all numbers in this description indicating amounts of material ought to be understood as modified by the word “about”.

The term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above.

The following examples will more fully illustrate the embodiments of this invention. All parts, percentages and proportions referred to herein and in the appended claims are by weight unless otherwise illustrated.

### EXAMPLE 1

Typical of the present invention is a first composition in the form of a gel and a second composition in the form of a paste. Each of these formulations is held in a separate compartment of a dual-compartment dispenser similar to that disclosed in U.S. Pat. No. 5,038,963 (Pettengill et al.).

TABLE I

| | |
|---|---|
| INGREDIENT | WEIGHT % |

Gel Composition 1A

| | |
|---|---|
| Glycerin | 41.33 |
| Pluracare ® F-127 ® | 20.00 |
| Hydrogen Peroxide (2.5% Active) | 4.285 |
| Polyethylene Glycol | 3.67 |
| Phosphoric Acid (85% Active) | 0.30 |
| Water | Balance |

Paste Composition 1B

| | |
|---|---|
| Polyol II (70% Sorbitol) | 45.53 |
| Tixosil 63 ® (Hydrated Silica) | 17.21 |
| Sodium Carbonate | 6.00 |
| Sodium Bicarbonate | 1.00 |
| Tixosil 43 ® | 3.00 |
| Ethyl Alcohol 38B | 2.84 |
| Sodium Lauryl Sulphate (SLS) | 2.98 |
| Flavor | 1.30 |
| Cellulose Gum | 0.80 |
| Sodium Saccharin | 0.54 |
| Menthol | 0.50 |
| Titanium Dioxide | 0.30 |
| Water | Balance |

### EXAMPLE 2

The compositions prepared in Example 1 were measured for their viscosity.

A Brookfield LV Viscometer was utilized to measure these viscosities. They were taken with a T bar (either C, D or E) operating at 50 rpm at 20° C. Measurement results are reported in the Table II.

TABLE II
Brookfield Viscosity Values

| FORMULATION | VISCOSITY (cps) |
|---|---|
| Bicarbonate Paste Composition 1B (with SLS)¹ | 66,500 |
| Bicarbonate Paste Composition 1B (no SLS)¹ | 48,000 |
| Gel² Composition 1A | 18,200 |
| Bleaching Composition (paste/gel mixture) | |
| (with SLS)³ | |
| Initial Viscosity | 6,200 |
| 10 Minute Viscosity | 6,600 |
| Bleaching Composition (gel/paste mixture) | |
| (no SLS)³ | |
| Initial Viscosity | 11,200 |
| 10 Minute Viscosity | 10,700 |

¹Measured with T bar E
²Measured with T bar D
³Measured with T bar C

Inclusion of SLS (sodium lauryl sulphate), a powder material, to the bicarbonate paste composition increased the viscosity of that component. However, the presence of SLS in the combined bleaching composition advantageously lowered viscosity (circa 6,200-6,600 cps) relative to one without SLS (11,200-10,700 cps). It was further noticed that the SLS containing bleaching composition mixture continued to bubble for 10 minutes whereas the SLS-free bleaching mixture did not bubble at all. The results of the presence of this anionic surfactant allowed for a more mobile bleaching composition and thereby was better able to penetrate all areas around the teeth.

### EXAMPLE 3

Another system typical of the present invention is a gel and paste along with Triclosan solubilized in alcohol. Each of these formulations is held in a separate compartment of a dual-compartment dispenser similar to that of Example 1.

TABLE III

| | |
|---|---|
| INGREDIENT | WEIGHT % |

Gel Composition 2A

| | |
|---|---|
| Glycerin | 40.00 |
| Pluracare ® F-127 ® | 20.00 |
| Ethyl Alcohol 38B | 5.00 |
| Phosphoric Acid | 0.45 |
| Hydrogen Peroxide (35% Active) | 4.29 |
| Irgacure ® MP (Triclosan) | 0.67 |
| Water | Balance |

Paste Composition 2B

| | |
|---|---|
| Polyol II (70% Sorbitol) | 46.68 |
| Tixosil 63°® (Hydrated Silica) | 15.00 |
| Sodium Bicarbonate | 10.00 |
| Tixosil 43 ® (Hydrated Silica) | 6.00 |
| Polyethylene Glycol 1450 (PEG-32) | 3.00 |
| Ethyl Alcohol 38B | 2.84 |
| Sodium Lauryl Sulphate | 2.98 |
| Flavor | 1.10 |
| Cellulose Gum | 0.80 |
| Sodium Saccharin | 0.54 |
| Menthol | 0.50 |
| Sodium Fluoride | 0.44 |
| Titanium Dioxide | 0.30 |
| Water | balance |

The pH of 2A and 2B respectively are approximate 2.7 and 9.2

The foregoing description and examples illustrate selected embodiments of the present invention. In light thereof variations and modifications will be suggested to one skilled in the art, all of which are within the spirit and purview of this invention.

What is claimed is:

1. A kit for whitening teeth comprising:
(i) a U-shaped dental tray fittable over a person's teeth;
(ii) a bleaching composition formed from combining a first and second composition, the compositions being separately stored and comprising:
(a) a first composition comprising from about 0.1 to about 30% of a peroxide in a cosmetically acceptable carrier;
(b) a second composition comprising from about 0.1 to about 30% of an alkaline material in a cosmetically acceptable carrier, wherein at least one of the first and second compositions comprises an anionic surfactant;
(iii) written instructions for placing the bleaching composition within the U-shaped tray and applying the tray over the person's teeth for a length of time sufficient to whiten teeth.

2. The kit according to claim 1 wherein the anionic surfactant is present in an amount from about 0.1 to about 20% by weight of the bleaching composition.

3. The kit according to claim 1 wherein the anionic surfactant is a salt of lauryl sulphate or lauryl ether sulphate.

4. The kit according to claim 1 wherein the alkaline material comprises a mixture of bicarbonate and carbonate in a weight ratio of about 1:2 to about 1:100.

5. The kit according to claim 4 wherein the weight ratio is about 1:5 to about 1:10.

6. The kit according to claim 1 wherein the anionic surfactant is present in an amount from about 0.01 to about 20% by weight of the bleaching composition.

7. The kit according to claim 1 wherein the anionic surfactant is present in an amount from about 0.5 to about 10% by weight of the bleaching composition.

8. The kit according to claim 1 wherein the first and second compositions are delivered from a dual-chambered pump dispenser.

9. The kit according to claim 1 wherein viscosity of the bleaching composition ranges from about 2,000 to about 100,000 cps.

10. The kit according to claim 1 wherein the dental tray comprises an anterior curved wall, a posterior curved wall and a connector bridge between the two walls, and a handle projecting outwardly from the anterior curved wall.

11. The kit according to claim 7 wherein the handle is unitarily formed with the anterior curved wall.

12. A method for whitening teeth comprising:
(a) providing a U-shaped dental tray fittable over a person's teeth;
(b) placing a bleaching composition within the U-shaped tray, the bleaching composition formed from combination of a first and second composition, the first and second compositions being separately stored and comprising:
(a) a first composition comprising from about 0.1 to about 30% of a peroxide in a cosmetically acceptable carrier;
(b) a second composition comprising from about 0.1 to about 30% of an alkaline material in a cosmetically acceptable carrier, wherein at least one of the first and second compositions includes an anionic surfactant;
(c) applying the bleaching composition filled U-shaped tray over the person's teeth for a length of time sufficient to whiten the teeth.
